(19) United States
(12) Patent Application Publication
(10) Pub. No.: US 2009/0035281 A1
(43) Pub. Date: Feb. 5, 2009

(54) COMBINATION OF INTERLEUKIN-6 ANTAGONISTS AND ANTIPROLIFERATIVE DRUGS

(75) Inventors: Rocco Savino, Soverato (IT); Pierfrancesco Tassone, Cantanzaro (IT); Salvatore Venuta, Napoli (IT)

Correspondence Address: LUCAS & MERCANTI, LLP, 475 PARK AVENUE SOUTH, 15TH FLOOR, NEW YORK, NY, 10016, US

(73) Assignee: Universita Degli Studi "Magna Graecia" Di Catanzaro

(21) Appl. No.: 11/908,022

(22) PCT Filed: Mar. 7, 2006
(86) PCT No.: PCT/EP2006/060503

§ 371 Date: May 27, 2008

(30) Foreign Application Priority Data
Mar. 10, 2005 (IT) RM 2005 A 000103
Mar. 7, 2006 (EP) PCT/EP2006/060503

(51) Int. Cl. A61K 39/395; A61K 38/17; A61K 31/445; A61K 31/675; A61P 35/00; A61K 35/12; A61K 31/66; A61K 31/56

(52) U.S. Cl. 424/93.7; 514/178; 514/12; 424/158.1; 514/320; 514/108; 514/94

(57) ABSTRACT

The combination of an interleukin-6 (IL-6) antagonist and an antiproliferative drug is described. In its preferred embodiment, the present invention describes the combination of an IL-6 superantagonist, particularly a superantagonist totally incapable of binding gp130 and an antiproliferative drug belonging to the glucocorticoid class (SANT-7 and dexamethasone). The combination according to the present invention has shown surprising synergism in an animal model of multiple myeloma and the ability to overcome the resistance to the antiproliferative drug developed by myeloid cells. The combination according to the present invention is useful for the preparation of a medicament for the treatment of tumours, particularly IL-6-dependent tumours.

The present invention relates to the medical field, and in particular the present invention provides a new combination of drugs useful for the treatment of hyperproliferative diseases, such as haematological tumours, and particularly multiple myeloma.

### BACKGROUND TO THE INVENTION

Multiple myeloma (MM) is a haematological tumour characterised by the monoclonal expansion of monotypical plasma cells in the bone marrow (Hideshima, T., Anderson, K C; Nat. Rev. Cancer, 2002; 2:927-937). Despite all the therapies currently available, the median survival is 4.4-7.1 years (Sirohi, B., Powles, R.; Lancet, 2004; 363:875-887) and the disease relapses even after apparent complete remission, probably due to the inevitabile development of clones of resistant tumour cells (Hideshima, T., ibid). Recently, high-dose chemotherapy followed by autologous transplantation of stem cells has been proposed (Attal, M., et al.; New Engl. J. Med., 1996, 335:91-97). However, this type of therapy also fails to prevent fatal relapses.

Glucocorticoids, such as prednisone or dexamethasone, are extensively used in the treatment of multiple myeloma (Alexanian, R., et al.; Blood, 1983; 62:572-577; Alexanian, R., et al.; Blood, 1992; 80:887-890). Dexamethasone, alone or in combination with other chemotherapeutic agents, e.g. alkylating agents, is a very important active ingredient against multiple myeloma and is used in both traditional and innovative therapeutic protocols. However, blockade of the IL-6 signalling pathway appears to be essential for the effects mediated by dexamethasone (Hardin, J., et al.; Blood, 1994; 84:3063-3070), since induction of apoptosis of MM cells by dexamethasone requires the activation of signal transduction pathways that can be inhibited by IL-6 and are independent of the protein kinases activated by stress, also known as C-Jun aminoterminal kinases (SAPK/JNK) (Chauhan, D., et al., Oncogene, 1997; 15:837-843; Xu, F. H., et al.; Blood, 1998; 92:241-251). In addition, dexamethasone does not completely suppress the production of IL-6 by bone marrow stromal cells (BMSC), which, albeit in limited amounts, continue to produce the cytokine, thus counteracting the cell death induced by dexamethasone (Grigorieva, L, et al.; Exp. Hematol., 1998; 26:597-603). The fact that dexamethasone only partially inhibits but does not abolish the production of IL-6 may explain why, despite the substantial response of multiple myeloma to the glucocorticoid drug, the MM cells develop drug resistance and the treatment fails to significantly increase long-term survival.

There is, therefore, a strongly perceived need to develop new treatments that overcome the limitations of the therapeutic strategies currently available. In particular, the problem of an effective therapy that prevents or attenuates the drawback of the onset of drug resistance by the tumour cells has yet to be solved. Furthermore, it should also be borne in mind that an effective antiproliferative therapy must also be selective, that is to say, it must not present substantial or major toxic effects on healthy cells.

Interleukin-6 (IL-6) plays an important role in multiple myeloma (Klein, B., et al.; Blood, 1995; 85:863-872; Hallek, M., et al.; Blood, 1998; 91:3-21). The physiological production of IL-6 induces the differentiation of normal plasmablastic cells into mature plasma cells secreting immunoglobulins (Bauer, J., Herrmann, F.; Ann. Hematol., 1991; 62:203-210; Akira, S., et al.; Adv. Immunol., 1993; 54.1-78). It has been demonstrated by several authors that IL-6 is one of the main growth factors for the malignant counterpart of plasma cells (Klein, B., et al.; Blood, 1995; Klein, B., et al.; Blood, 1989; 73:517-526). The myeloma cells that express a functional IL-6 receptor (Klein, B., et al.; Blood, 1989; 73:517-526; Klein, B.; Semin. Hematol., 1995; 32:4-19) depend on IL-6 for growth, and their proliferation is inhibited by anti-IL-6 antibodies (Klein, B., et al., Blood, 1989; 73:517-526). The in-vivo administration of anti-IL-6 monoclonal antibodies (mAb) causes cytostatic effects on tumour cells (Bataille, R., et al.; Blood, 1995; 86:685-691). An important element for establishing an effective therapy for multiple myeloma is provided by IL-6 antagonism of cell death by apoptosis induced in multiple myeloma by a series of active ingredients, including dexamethasone (Dex); thus, an IL-6 antagonist might be potentially useful in the therapy of multiple myeloma (Hardin, J., et al.; Blood, 1994; 84.3063-3070; Shiao, R. T., et al., Leuk. Lymphoma, 1995; 17:485-494).

Molecular variants of IL-6 have been produced that bind with high affinity for the IL-6R alpha chain and prevent the generation of the binding and/or dimerisation of the gp130 transducing chain (Savino, R., et al.; Embo J., 1994; 13:1357-1367; Sporeno, E., et al.; Blood, 1996; 87:4510-4519; Demartis, A., et al.; Cancer Res., 1996; 56:4213-4218; WO 96/34104). Of these variants, the most potent belong to a series of superantagonists of human interleukin-6, completely incapable of binding gp130, described in the above-cited WO 96/34104, a representative member of which is known by the name of SANT-7. The latter exerts strong inhibition of cell proliferation and is endowed with substantial efficacy as a proapoptotic factor for IL-6-dependent multiple myeloma cells. It has also been demonstrated that SANT-7 is capable of overcoming IL-6-mediated cell resistance to dexamethasone in an autocrine setting (Tassone, P., et al.; Cell Death Differ., 2000; 7:327-328). In this latter study, only an in-vitro model was given and no animal model was indicated for the necessary in-vivo verification. Moreover, this study does not analyse the effect of the micromilieu of human bone marrow. This effect is studied in a later paper (Hönemann, D., et al.; Int. J. Cancer, 2001, 93:674-680), demonstrating that, unlike the study by Tassone et al. in Cell Death Differ., not even SANT-7 manages to confirm its potent activity originally demonstrated in vitro on human multiple myeloma cells in single culture, but only the combination of SANT-7 and a chemotherapeutic agent is capable of overcoming the drug resistance of the MM cells induced by Il-6 secreted in the micromilieu of the bone marrow. In this study, the authors, including the present inventors, conclude that the relevance of IL-6 for the growth, survival and drug resistance of multiple myeloma cells in vivo is not entirely clear and they suggest that the possibility of combining SANT-7 with other drugs might be a useful approach to the treatment and might make an interesting contribution to the understanding of myeloma. In this case, too, no indications are provided that may be useful for testing the hypothesis in a validly accepted animal model of human multiple myeloma, not even that this hypothesis may have a reasonable prospect of success. The strengthening in vitro of the antimyeloma activity of SANT-7 has also been demonstrated for the combination of dexamethasone and zoledronic acid (Tassone, P., et al.; Int. J. Oncol., 2002; 21:867-873), suggesting that inhibition of the IL-6 survival pathway may effectively be a valid antimyeloma strategy. The authors, including the present inventors, have attempted to provide an in-vitro model that resembles the situation in vivo, where the growth of the MM cells is influenced by both autocrine and paracrine IL-6, administering IL-6 to cell cultures. In another in-vitro model, the effect on primary bone marrow MM cells (Bone Marrow cultures, BMc) was assessed. Apart from the difficulty of reliably measuring IL-6 in the supernatants of samples with SANT-7, the synergism of the triple combination was not always confirmed. The lack of reliable measurement of IL-6 levels does not allow proper evaluation of SANT-7 activity, leaving in some doubt the issue as to whether the molecule effectively works or whether the assay is not appropriate, thus necessitating long and difficult experimentation. Furthermore, the effect of adhesion of the MM cells to the bone marrow cells was not evaluated. However much the authors may encourage this type of combination therapy, no valid in-vivo experimental model is indicated.

Dexamethasone, alone or in combination with other drugs, is an active ingredient used in the treatment of multiple myeloma (Alexanian, R., et al.; Blood, 1983; 62:572-577; Alexanian, R., et al.; Blood, 1992; 80:887-890). However, the paracrine secretion of IL-6 by the BMSCs in the micromilieu of the bone marrow, greatly increased by the adhesion of the MM cells (Uchiyama, H., et al.; Blood, 1993; 82:3712-3720; Caligaris-Cappio, F., et al; Blood, 1991; 77:2688-2693; Lokhorst, H. M., et al.; Blood, 1994; 84:2269-2277), leads to the accumulation of fairly substantial amounts of the cytokine which counteracts the antimultiple-myeloma effects induced by dexamethasone (Hardin, J., et al.; Blood, 1994; 84:3063-3070). The therapeutic activity of dexamethasone might therefore hypothetically be increased by combination with factors capable of neutralising the effects of IL-6. To this end, various biological substances were used in the past (Portier, M., et al.; Blood, 1993; 81:3076-3082; Schwabe, M., et al.; J. Clin. Invest., 1994; 94:2317-2325; Herrmann, F., et al., Blood, 1991; 78:2070-2074; Levy, Y, et al.; Clin. Exp. Immunol., 1996; 104:167-172), including anti-IL-6 monoclonal antibodies (Bataille, R., et al.; Blood, 1995; 86:685-691). In actual fact, the anti-IL-6 monoclonal antibodies proved effective only transitorily and partially, owing to the difficulty of blocking large amounts of 11-6. Furthermore, anti-IL-6 monoclonal antibodies also have a “paradoxical” effect, in that it has been demonstrated that they stabilise the cytokine in the form of circulating Il-6/antibody complexes, which in contrast to the very short half-life of the soluble cytokine (Castell, J. V, et al.; Eur. J. Biochem., 1988; 177: 357-361), have a half-life of 3-4 days in vivo (Lu, Z. Y, et al.; Eur. J. Immunol., 1992; 22: 2819-2824), thus contributing to the accumulation of the circulating cytokine which is then released with devastating effects when the treatment with the monoclonal antibody is discontinued (Klein, B. et al.; Blood, 1991; 78:1198-1204).

The recombinant IL-6 receptor antagonists, which bind to the IL-6R alpha chain, inhibit the assembly of the functional complexes of the IL-6 receptor (Savino, R., et al.; Embo J. 1994; 13:1357-1367; Sporeno, E., et al.; Blood, 1996; 87:4510-4519; Demartis, A., Cancer Res., 1996; 56:4213-4218), and present the considerable advantage of efficiently and selectively inhibiting the transduction of the IL-6-mediated signal without affecting other signal pathways in the target cell. These compounds, and particularly the IL-6 receptor superantagonist SANT-7, have been shown to block the IL-6 signal and induce a high mortality in the IL-6-dependent MM cells (Demartis, A., et al.; Cancer Res., 1996; 56:4213-4218). SANT-7 may therefore be a suitable agent for use in combination with other drugs in the treatment of MM. It has previously been reported that the treatment of an MM cell line partly dependent on IL-6 with SANT-7 can overcome the IL-6-mediated cell resistance to dexamethasone, giving rise to the specific depletion of the MM cell population in cocolture with primary CD34⁺ HPC (Tassone, P., et al.; Cell Death Differ., 2000; 7:327-328), suggesting that a combined approach to multiple myeloma might advantageously utilise such agents.

Previous experience with combinations of glucocorticoid drugs and substances capable of neutralising IL-6, such as SANT-7, have never yielded coherent, encouraging results for the clinical development of any such combination. In point of fact, the in-vivo models, none of which are representative of a model of human multiple myeloma, have never confirmed the in-vitro data, nor provided an acceptable scientific basis allowing the expert in the field to undertake onerous clinical trials with any reasonable expectation of success. Therefore, the expert in the field would not have been able to draw definitive conclusions regarding the therapeutic effect of a hypothetical combination of dexamethasone and SANT-7.

Animal models can usually provide important information regarding human diseases. Human B dell lines grow easily in mice with severe combined immunodeficiency (SCID). Such mice have a severely impaired immune system and are capable of accepting extraneous cells. Nevertheless, plasma cells explanted from patients with multiple myeloma and IL-6-dependent myeloma cell lines do not grow in mice. The difficulty in growing human myeloma cell lines in mice reflects the dependence of the human myeloma plasma cells on the micromilieu of the bone marrow, which assists their growth. This critical requirement of human myeloma cells cannot be replaced by the micromilieu of murine bone marrow.

Therefore, finding a solution to the problem of the development of drug resistance by means of a hypothetical combination of currently used drugs and some substance capable of interfering with the IL-6-mediated signalling pathway was effectively impeded by the unavailability of a valid animal model allowing the expert in the field to have the necessary confirmation of the experimental data available obtained from in-vitro models. The lack of such a model constitutes an effective impediment to designing the clinical development of the hypothetical combination, given that the expert in the field does not have all the information and instructions needed to allow him to conduct the necessary preclinical experiments so as to be able then to undertake clinical trials in human subjects, which are much more onerous not only from the economic point of view, but above all from the ethical standpoint. In fact, regulatory authorities will not authorise the start of clinical trials without valid preclinical experimentation that indicates the possibility of therapeutic success with reasonable certainty. Thus, the previous tests of the hypothetical combination of a drug useful in the treatment of multiple myeloma and a substance capable of inhibiting the actual signalling pathway of interleukin-6 are not regarded as sufficient and complete by the experts in the medical field.

### SUMMARY OF THE INVENTION

A new animal model has now been found which has enabled the present inventors to validate scientifically the efficacy of a combination of drugs traditionally used in the treatment of multiple myeloma and substances that interfere with the IL-6 signalling pathway.

Thanks to this model it has unexpectedly proved possible to find a surprising synergistic effect in vivo between the substance interfering with the IL-6 signalling pathway, in particular, a superantagonist of human interleukin-6, totally incapable of binding gp130, and an anti-proliferative drug such as a glucocorticoid. The synergistic effect is totally unexpected on the basis of what is known about cytokines and SANT-7. The cytokines have an extremely rapid kinetics, and thus finding the antagonist effect in vivo was thoroughly unexpected. In the course of the studies that led to the present invention, the pharmacokinetics of SANT-7 was seen to be very rapid, and therefore the drug does not present a very favourable profile for combination therapy in long-term treatment. In fact, in subcutaneous administration (one of the preferred routes in the case of proteins) it presents a very rapid clearance and would require frequent administrations. Thus, the expert in the field would not have found any reason to feel encouraged to design a therapy for a very long-term treatment with a drug of the SANT-7 type against an elusive target, such as IL-6. On the contrary, however, this combination provides a solution to the problems of the state of the art. Therefore, one object of the present invention is a combination of an antiproliferative drug and an antagonist, particularly a superantagonist, of the interleukin-6 receptor.

Another object of the present invention is the use of said combination for the preparation of a medicament useful for the treatment of tumours. A further object of the present invention consists in pharmaceutical compositions containing said combination.

Advantageously, such a combination increases the effects of the anti-proliferative drug and counteracts the paracrine action of IL-6 in supporting the survival of the tumour cells. Therefore, a particular object of the present invention is the use of said combination for the preparation of a medicament useful for the treatment of IL-6-dependent tumours.

It has proved possible to demonstrate this through the use of a new murine model of human multiple myeloma.

The present invention will now be illustrated in detail by means of the following description, as well as by means of examples and figures, in which:

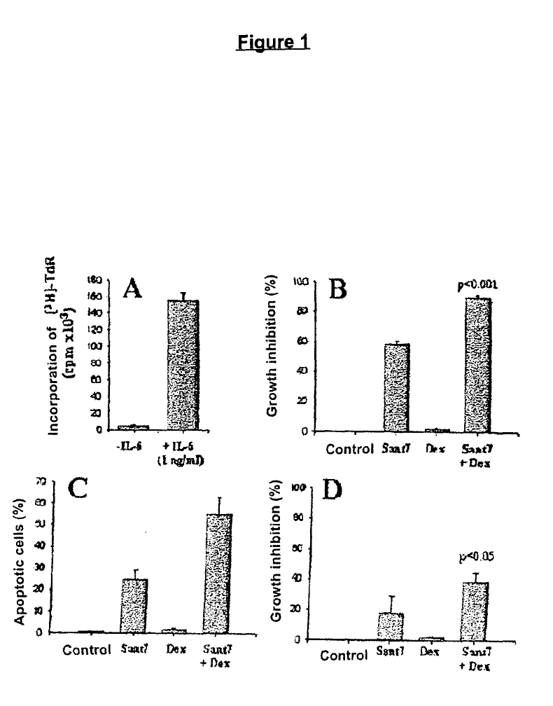

FIG. 1 shows the in-vitro effects induced by SANT-7 and/or dexamethasone (Dex) on the human IL-6-dependent multiple myeloma (MM) cell line, INA-6, after 3 days' culture. A) Cell proliferation in the presence or absence of exogenous IL-6, as determined by incorporation of [³H]-TdR. B) Growth inhibition effect of SANT-7 and/or Dex in cell cultures in the presence of exogenous IL-6; the data are expressed as percentages of the control values by measuring the incorporation of [³H]-TdR. C) Apoptotic effects induced in cell cultures in the presence of exogenous IL-6. Apoptotic cell death was determined by flow cytometry analysis of annexin V and staining with propidium iodide (PI). D) Growth inhibition effect on MM cells adhering to BMSC in the absence of exogenous IL-6. The growth inhibition effect was calculated as a percentage of the control value.

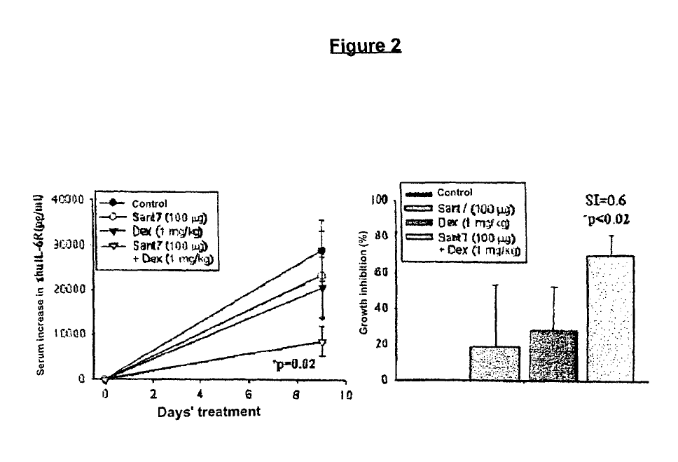

FIG. 2 shows the in-vivo kinetics of SANT-7 and the effects of SANT-7 and/or Dex in a new murine model of human MM in SCID-hu mice. A) Sant-7 (3.3 mg/kg) was injected s.c. into a SCID-hu mouse and its kinetics was evaluated with serial determinations of IL-6 in serum. B) Tumour growth in SCID-hu mice implanted with INA-6 cells was monitored with serial measurements of shuIL-6R. The antitumour effects were determined after 6 consecutive days of treatment s.c. with SANT-7 (3.3 mg/kg) and/or Dex (1 mg/kg). The groups of mice were: control (n=7), and cohorts treated with SANT-7 (n=4), Dex (n=4), and SANT-7 plus Dex (n=4). P values were obtained by comparison between the control groups and the groups treated with the combination. The data were expressed as mean±SE.

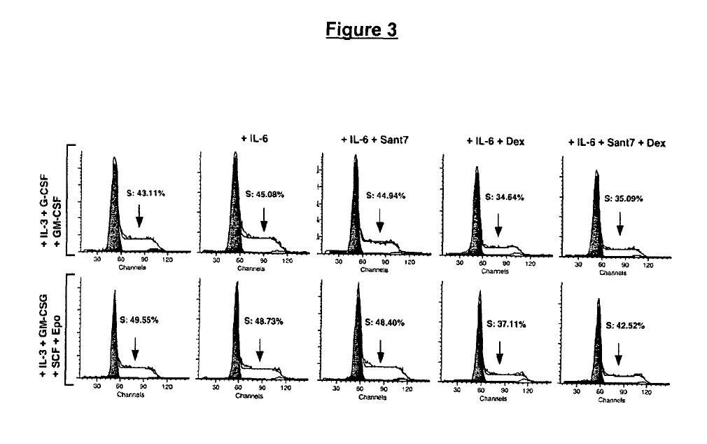

FIG. 3 shows the analysis of the cell cycle pf HPC exposed to SANT-7 and/or Dex. Flow cytometry profile of a representative experiment in which the cell cycle is analysed by means of staining with PI. The analysis was carried out with a Cell-Quest program (Becton Dickinson). The treatments and percentages of cells in phase S are indicated.

### DETAILED DESCRIPTION OF THE INVENTION

The substances that interfere with the signalling pathway mediated by interleukin-6 are a family of superantagonists of human interleukin-6.

In a preferred embodiment of the invention, the superantagonist is totally incapable of binding gp130 and is selected from the group consisting of the proteins described by the respective sequences SEQ ID No. 1, SEQ ID No. 2, SEQ ID No. 3 and SEQ ID No. 4. Among these, the one preferred is the protein called SANT-7, described by the sequence SEQ ID No. 4.

Falling within the scope of the present invention are those mutants of IL-6 superantagonists, and particularly those that are totally incapable of binding gp130, which in the first place maintain their 11-6 antagonist capacity and, secondly, their ability not to bind gp130. The conformity of the mutant in the context of the present invention can be determined using the methods described in the above-mentioned WO 96/34104.

The family of superantagonists of human interleukin-6 totally incapable of binding gp130 is described in the international patent application WO 96/34104 and subsequent publications by the inventors (Savino, R., et al.; Embo J. 1994; 13:1357-1367; Sporeno, E., et al.; Blood. 1996; 87:4510-4519; Demartis, A., et al.; Cancer Res., 1996; 56:4213-4218)

Equally well known are the gene sequences that code for the above-mentioned superantagonist proteins, as described in the above-cited international patent application. Therefore, a further object of the pre-sent invention is the use of gene sequences that code for the proteins described by the respective sequences from SEQ ID. No 1 to SEQ ID No. 4 for the preparation of a medicament useful in gene therapy. In this context, the gene therapy consists in administering the sequence selected, achieving expression of the corresponding protein, and, once the protein has exerted its anti-Il-6 antagonist action, administering the antiproliferative drug. The therapeutic indications are the same as for the combination of the protein and the antiproliferative drug. The administration of the gene sequence and its expression in the protein are done using conventional techniques. An example of such techniques is described in the publications of one of the present inventors (Savino) on the development of adenoviral vectors; see, for example, U.S. Pat. No. 6,641,807 and U.S. Pat. No. 6,475,755.

The present invention will now be described in one of its preferred embodiments, that is to say, in the use of the combination for the preparation of a medicament useful for the treatment of human multiple myeloma, on the basis of the specifically developed animal model.

This embodiment does not rule out the possibility of implementing the invention also for the treatment of other diseases, such as interleukin-6-dependent tumours.

The combination according to the present invention can also be used in other multiple myeloma therapies, particularly for overcoming the drug resistance developed by the multiple myeloma cells. The combination according to the present invention can be used in all therapies that employ glucocorticoids, either alone or in combination with therapies involving biological agents or conventional forms of chemotherapy, for example, those which use or which could use alkylating agents such as melfalan, all-transretinoic acid, thalidomide, and biphosphonates such as zoledronic acid. In a preferred embodiment of the invention, the combination is used in conjunction with zoledronic acid. The combination can also be used in conjunction with therapies involving high-dose chemotherapeutic treatment followed by autologous stem cell transplantation.

It is interesting to note that these active ingredients, whether alone or in combination, do not interfere significantly with CD34⁺ growth and survival.

A further advantage of the combination according to the present invention is that the therapeutic effect is boosted, without additional adverse effects on the haematopoietic progenitor cells.

The in-vivo model, specifically developed for the combination which is the object of the present invention, includes the injection of Il-6-dependent INA-6 cells in a human foetal bone implant in SCID mice. The human foetal bone implant in SCID mice supports the growth of primary human myeloma cells and the proliferation of the myeloma cells produces the typical manifestations of the disease, such as the increase in levels of monoclonal Igs, and the reuptake of human bone, reproducing human myeloma. It is interesting to note that myeloma cells do not grow in mice and remain confined to the human bone. If the human bone is implanted in the other flank, the cells migrate to that bone without growing in the mouse bone. These SCID-hu mice will be a useful model for studying the in-vivo effects of various new compounds in multiple myeloma in an effort to find an effective therapeutic combination.

For the first time, the present invention provides evidence that the combination of a superantagonist of interleukin-6, particularly the one known as SANT-7, and an antiproliferative drug, such as dexamethasone, exerts an unexpected synergistic effect in the treatment of tumour forms, such as multiple myeloma.

To the best of the inventors' knowledge, this is the first in-vivo experimental demonstration of a synergistic action of interleukin-6 and a glucocorticoid.

The following example further illustrates the invention.

### EXAMPLE

INA-6 cells were cultured either in the presence of exogenous IL-6 or adhering to bone marrow stromal cells (BMSC), with SANT-7 and/or dexamethasone (Dex). The in-vitro effects were determined by measuring cell proliferation and/or apoptosis. The in-vivo effects induced by these drugs were then studied in a murine model of human MM, in which the cells were injected directly into human bone marrow implants in SCID (SCID-hu) mice. The in-vivo treatments were monitored with determination of the soluble 11-6 receptor (shuIL-6R) in serum, which is released by INA-6 cells. The effects induced by both drugs on CD34⁺ haematopoietic progenitor cells were examined.

The in-vitro treatment of INA-6 cells with SANT-7, in the presence of exogenous IL-6, induced a high rate of inhibition of cell growth and a high rate of apoptotic cell death in MM cells. Exogenous IL-6 completely inhibited the effects induced by Dex. The combination of SANT-7 and Dex gave rise to a synergistic anti-MM effect. Adherence of the INA-6 cells to BMSC reduced the activity of SANT-7 and inhibited the effects induced by Dex. However, also in the case of cells adhering to BMSC, the combination of SANT-7 and Dex gave rise to synergistic effects. In SCID-hu mice, treatment with SANT-7 or Dex alone was well tolerated, but did not produce any significant reduction in serum levels of shuIL-6R. In contrast, the SANT-7 plus Dex combination gave rise, after 6 consecutive days' treatment, to a synergistic level of inhibition of tumour growth, that is to say, the effect is unexpectedly greater than the expert in the field might expect on the basis of his knowledge of the two individual drugs. In-vitro assays on the colonies showed weak inhibition of the generation of myeloid and erythroid colonies by normal CD34⁺ progenitor cells in response to Dex, whereas SANT-7 showed no intrinsic activity and did not even enhance the inhibitory action of Dex on the differentiation of progenitor cells.

The inhibition of the IL-6 signal transduction pathway by an IL-6 antagonist significantly enhances the therapeutic action of Dex against MM cells both in vivo and in vitro, at doses well tolerated in mice.

The superantagonist of the IL-6 receptor, SANT-7, was prepared according to the procedure described in WO 96/34104 and in Savino, R., et al.; Embo J. 1994; 13:1357-1367; Sporeno, E., et al.; Blood. 1996; 87:4510-4519; Demartis, A., et al.; Cancer Res., 1996; 56:4213-4218.

SANT-7 is a molecular variant of IL-6 which binds with high affinity to the IL-6R alpha chain and prevents the binding and dimerisation of the gp130 chain, inhibiting the transduction of the signal produced by IL-6. All the reagents are available on the market or can be obtained using methods described in the literature. In the case of the present example, dexamethasone is the speciality Soldesam® from American Pharmaceutical Partners, Inc, Schaumburg, Ill., USA; IL-6, IL-3, stem cell factor (SCF), and the ligand FLt3 (FL) are from PeproTech EC Ltd (London, UK). Granulocyte colony-stimulating factor (G-CSF) and erythropoietin (Epo) are from Dompe-Biotec (Milan, Italy). Granulocyte-macrophage colony-stimulating factor (GM-CSF) is from Schering-Plough (Milan, Italy); anti-CD34 (HPCA-2) is from Becton Dickinson (San Jose, Calif., USA).

The formation, characterisation and in-vitro culturing of the IL-6-dependent human MM cell line INA-6 is described in Burger, R., et al.; Hematol. J., 2001; 2:42-53. The cells were maintained in RPMI 1640 culture medium (GIBCO, Grand Island, N.Y.) added with 10% foetal calf serum (FCS, Hyclone, Logan, Utah), L-glutamine 2 mM (GIBCO), 100 μg/ml of streptomycin (GIBCO) and 100 U/ml of penicillin (GIBCO) in the presence of 2.5 ng/ml of IL-6 at 37° C. in a 5% CO₂ atmosphere

Peripheral blood mobilised CD34⁺ HPC were isolated from leukapheresis products of patients with haematopoietic and non-haematopoietic tumours, treated with high-dose chemotherapy and G-CSF or GM-CSF. Peripheral blood mononuclear cells were obtained by centrifugation across a Ficoll density gradient (Seromed, Berlin, Germany), washed and submitted to positive selection using the CD34 Progenitor Cell Isolation Kit (Miltenyi Biotech, Bergish Gladbach, Germany). In brief, CD34⁺ HPC were magnetically labelled indirectly using a primary monoclonal antibody conjugated with a hapten and an anti-hapten antibody coupled with MACS MicroBeads (Miltenyi). The labelled cells were subsequently enriched with the MiniMACS magnetic field. The purity of the CD34⁺ HPC isolated was generally above 85%, as determined by flow cytometry (Coulter, Birmingham, UK); cell viability was evaluated by cell staining with PI and exclusion of tryptan blue, and was usually >90%.

### Cell Proliferation Assay

Cell proliferation was measured by incorporation of [³H]-thymidine (NEN Life Science Products, Boston, Mass.). Cells (2×10⁴ cells/well) were incubated on 96-well culture plates in the presence or absence of 70-80% confluent BMSC at 37° C. with or without the study substance (in wells in triplicate) for 72 h. [³H]-thymidine (0.5 μCi) was then added to each well for at least 8 h. Cells were collected on glass filters with an automatic cell collector (Cambridge Technology, Cambridge, Mass.) and counted using a Micro-Beta Trilux counter (Wallac, Gaithersburgh, Md.).

### Detection of Apoptosis

To detect the induction of cell death by apoptosis, double staining was performed with annexin V labelled with FITC and propidium iodide (PI). After treating 1×10⁶ tumour cell for 48 h, the cells were washed with PBS and resuspended in 100 μl of HEPES buffer containing annexin V-FITC and propidium iodide (PI) (Annexin V-FLUOS staining kit; Roche Diagnostic, Indianapolis, Ind.). After 15 minutes' incubation at room temperature, the cells were analysed using a Coulter Epics XL flow cytometer to detect the presence of an apoptotic cell population staining positive for annexin V-FITC and negative for PI.

### SCID-hu INA-6 Mouse Model

Male SCID C-17 mice aged from six to eight weeks (Taconic Germany, N.Y.) were housed and monitored in our Animal Research Facility. All the experimental procedures and protocols were approved by the Institutional Committee on the Treatment and Use of Animals. Human foetal femur transplants were implanted in SCID (SCID-hu) mice, as described in Urashima, M., et al.; Blood, 1997; 90(2): 754-65; Tassone, P., et al.; Blood, 2004. Four weeks after implantation, 2.5×10⁶ INA-6 MM cells in 50 μl of PBS were injected into the foetal bone implant in the SCID-hu hosts. Serum levels of the interleukin-6 soluble receptor (shuIL-6R) (R & D Systems Inc., Minneapolis, Minn.) were monitored in the mice.

Liquid culture of human CD34⁺ Haematopoietic Progenitor Cells (HPC)

Isolated CD34⁺ HPC were cultured at a density of 1×10⁵ cells/well on 24-well plates (Falcon, Becton Dickinson Labware, Frankil Lakes, N.J.) in 1 ml of Dulbecco culture medium modified according to Iscove (IMDM) (GIBCO) added with 10% foetal calf serum (Hyclone) and 1% deionised bovine serum albumin (Sigma, St Louis, Mo., USA). To induce granulomonocytic or erythroid differentiation, the cells were stimulated with IL-3 (50 ng/ml), GM-CSF (100 ng/ml), G-CSF (100 ng/ml) or IL-3 (50 ng/ml), GM-CSF (100 ng/ml), SCF (50 ng/ml) and Epo (3 U/ml), respectively. When indicated, the cells were also cultured in the presence of IL-6 (0.2 ng/ml) with the addition of SANT-7 (200 ng/ml) and/or Dex (10−5 M) to study the effect of these molecules on the cell cycle and differentiation. The cultures were maintained in a 5% CO₂ humidified atmosphere in air at 37° C. and were collected on day 6. Cell viability was determined by means of tryptan blue exclusion.

### Clonogenic Progenitor Assays

The clonogenic progenitor assays were carried out in methylcellulose as described previously with minor modifications. In brief, 1×10³ freshly isolated CD34⁺ HCP were seeded in IMDM (GIBCO) containing 1% methylcellulose, 30% foetal calf serum (Hyclone), 1% bovin serum albumin (Sigma), L-glutamine 2 mM (GIBCO) and 2β-mercaptoethanol 10⁻⁴ M (Stemcell Technologies Inc., Vancouver, Canada). To induce granulomonocytic or erythroid differentiation, the cells were stimulated with IL-3 (50 ng/ml), GM-CSF (100 ng/ml), G-CSF (100 ng/ml) or IL-3 (50 ng/ml), GM-CSF (100 ng/ml), SCF (50 ng/ml) ed Epo (3 U/ml), respectively. When indicated, IL-6 (0.2 ng/ml), SANT-7 (200 ng/ml) and/or Dex (10−5 M) were added to the cultures. 1 ml aliquots were plated in triplicate on 35 mm culture plates (Falcon) at 37° C. in a 5% CO₂ humidified atmosphere. After 14 days' culture, the granulomonocytic colonies (CFC-GM) and the erythroid colonies (BFU-E) were counted by examining the cultures under an inverted microscope.

### Statistical Analysis

The results were expressed as mean±SE. The statistical significance of the differences between the experimental points for single and combined treatment was analysed using the t-test; differences were considered significant when the P value was <0.05.

### Results

### The Combination of SANT-7 and Dex Induces Synergistic Anti-MM Effects In Vitro

IL-6 has been identified as one of the main factors in the growth and survival of MM cells. INA-6 is a human myeloma cell line that requires exogenous IL-6 for growth in vitro (FIG. 1A). This cell line was used to assess the effects induced by SANT-7 and/or Dex on the in-vitro growth of MM cells. INA-6 cells were seeded and cultured on 96-well plates in the presence of exogenous IL-6, and then, after 3 days' exposure of the cells to the drugs, cell proliferation and apoptosis were determined. Elimination of the IL-6 signalling pathway by SANT-7 induced high rates of growth inhibition and death by apoptosis in MM cells (FIGS. 1B and C). Dex alone neither modified cell proliferation nor induced apoptosis. By contrast, the combination of SANT-7 and Dex gave rise to synergistic antiproliferative and apoptotic effects, inhibiting the growth and survival of almost all the MM cells.

Since the paracrine production of IL-6 occurs when the MM cells adhere to BMSC (Uchiyama, H., et al.; Blood, 1993; 82:3712-3720), the supporting effect of BMSC for the in-vitro growth of INA-6 cells after exposure to the drugs was evaluated. The INA-6 cells were seeded on 70-80% confluent BMSC, in the absence of exogenous IL-6, and the cell proliferation was established 3 days after the treatment. As shown in FIG. 1D, the adherence of the INA-6 cells to the BMSC reduced the efficacy of the growth inhibition exerted by SANT-7, as compared with the cultures not adhering to the BMSC in the presence of exogenous IL-6. Dex activity was inhibited. The combination of the two agents still exerted significant, synergistic growth inhibition (P<0.05)

### SANT-7 Increases the Inhibition of Growth Induced by Dex In Vivo in a SCID-hu Model of Human MM

To evaluate the in-vivo effect of the combination of SANT-7 and Dex on MM cells in a human bone marrow milieu, a new murine model of human MM was used in which the INA-6 cells were directly injected into a piece of human foetal bone previously implanted in an SCID (SCID-hu) mouse. In these mice serum shuIL-6R was measured as a marker of tumour growth and disease severity, since it is released by the INA-6 cells. First, the pharmacokinetics of SANT-7 was determined. As shown in FIG. 2, after a single injection of SANT-7 (3.3 mg/kg), the SANT-7 serum peak was rapidly reached after 30 min, with the remaining drug in circulation for 4 hours after the injection. A cohort of 19 SCID-hu mice, previously transplanted with INA-6 cells s.c., were treated with SANT-7 and/or Dex for 5 consecutive days, and serial determinations of serum levels of shuIL-6R as a marker of tumour growth were performed. As shown in FIG. 2B, the treatment of SCID-hu mice with SANT-7 (3.3 mg/kg; n=4) or Dex alone (1 mg/kg; n=4) did not induce any significant reduction in shuIL-6R (P=0.5 and p=0.3, respectively) in comparison with the control group (PBS; n=7). In contrast, despite the relatively rapid pharmacokinetics of the recombinant protein, the combination of SANT-7 (3.3 mg/kg) and Dex (1 mg/kg) (n=4) reduced shuIL-6R levels significantly (P=0.04) and synergistically by up to 70% compared to the control group.

Effect of SANT-7 and/or Dex on Human HPC

To evaluate the safety of the SANT-7 plus Dex combination for clinical use, particularly in a post-transplant situation, the effects induced by these drugs on HPC were also evaluated. CD34⁺ cells purified by leukapheresis from cancer patients treated with high-dose chemotherapy and recombinant haemopoietins were exposed to SANT-7, alone or in combination with Dex, and analysed by clonogenic assays and flow cytometry. The results of the clonogenic assays (Table I) indicate that SANT-7 does not interfere appreciably with the generation of CFC-GM and BFU-E in response to haemopoietins. The addition of Dex, on the other hand, results in a reduction in the numbers of both types of colonies. SANT-7 does not enhance this inhibiting effect of Dex.

TABLE 1
Clonogenic assays of purified CD34+ HPC, carried out in semisolid
culture medium. The cells (1 × 10³/plate) were seeded in the presence
of haemopoietins to induce granulomonocytic (IL-3 + GM-CSF + G-CSF)
and/or erythroid differentiation (IL-3 + GM-CSF + SCF + Epo). The
cytokine concentrations used were: IL-3, 50 ng/ml; GM-CSF, 100 ng/ml;
G-CSF, 100 ng/ml; SCF, 50 ng/ml; Epo, 3 U/ml. The cultures were
counted on day 14. The data reported are mean ± SD of triplicates of a
representative experiment.

| Culture conditions | CFC-GM | BFU-E | Total |
|---|---|---|---|
| IL3 + GM + G | 90 ± 6 | | 90 ± 6 |
| +IL6 | 76 ± 1 | | 76 ± 1 |
| +IL6 + SANT-7 | 73 ± 2 | | 73 ± 2 |
| +IL6 + Dex | 54 ± 5 | | 54 ± 5 |
| +IL6 + SANT-7 + Dex | 55 ± 1 | | 55 ± 1 |
| IL3 + GM + SCF + Epo | 21 ± 3 | 75 ± 2 | 96 ± 2 |
| +IL6 | 30 ± 2 | 45 ± 1 | 75 ± 1 |
| +IL6 + SANT-7 | 21 ± 1 | 50 ± 2 | 71 ± 1 |
| +IL6 + Dex | 11 ± 2 | 29 ± 3 | 40 ± 2 |
| +IL6 + SANT-7 + Dex | 13 ± 2 | 37 ± 1 | 50 ± 1 |

Flow cytometric analysis of the DNA content was done on liquid cultures of CD34⁺ cells stimulated for 6 days with combinations of haemopoietins (IL-3+G-CSF+GM-CSF+IL-6 o IL-3+GM-CSF+Epo+IL-6) plus SANT-7, Dex or the combination of both drugs (FIG. 3). Whereas SANT-7 does not significantly affect cell proliferation, the addition of Dex causes a roughly 20% reduction in the number of cells in phase S. The combination of SANT-7 and Dex showed an effect similar to that of Dex alone. No significant apoptosis rate was detected.

As regards the aspects relating to industrial applicability, the combination according to the present invention can be conveniently formulated in a pharmaceutical composition. This composition may be a simple combination of known pharmaceutical forms of the individual active ingredients, the dosage of which will be established according to the modalities stemming from the application of the principles and instructions outlined in the present invention, that is to say, doses such as to ensure the reciprocal synergism. In that case, the composition according to the present invention may also be in the form of a kit, i.e., a pack grouping together the individual dosage forms of the active ingredients and the instructions for their simultaneous or sequential administration. Alternatively, the present invention provides for a new pharmaceutical composition containing the two active ingredients in a single dosage form. Advantageously, this dosage form will contain effective amounts of active ingredients such as to provide therapeutic cover with a minimal number of daily administrations. The doses and administration modalities will be established by the expert in the field, for example, the clinician or primary care physician, availing himself of his own general knowledge. On preferred example of a dosage form consists in a dose ranging from 1 mg to 1 g. The pharmaceutical compositions according to the present invention are thoroughly conventional and need no particular description. As regards the administration of the interleukin-6 receptor antagonist substance, since the latter is a peptide compound, the preferred administration forms will be parenteral. As is known, however, this substance can also be administered by the enteral route, particularly orally, using the methods commonly adopted for the preparation of gastroprotected formulations. In any event, a general description of pharmaceutical compositions is to be found in Remington's Pharmaceutical Sciences, latest edition, Mack Publishing and Co.

In the case of combination therapies, also with other drugs, the expert in the field can assess the suitability of variously combining the active ingredients, both in single dosage form and in the form of separate dosages, in which case the medicament may be in the form of a kit. The SEQ ID No 1, SEQ ID No 2, SEQ ID No 3 e SEQ ID No 4 sequences are given here below.

1. Combination of an antiproliferative drug and an interleukin-6 receptor antagonist.

2. Combination according to claim 1, in which said antagonist is a superantagonist totally incapable of binding gpl 30.

3. Combination according to claim 2, in which said superantagonist is a protein selected from the group described by the respective sequences SEQ ID No 1, SEQ ID No 2, SEQ ID No 3 and SEQ ID No 4.

4. Combination according to claim 3, in which said superantagonist is the protein called SANT-7, with sequence SEQ ID No 4.

5. Combination according to claim 1, in which said antiproliferative drug is a glucocorticoid.

6. Combination according to claim 5, in which said glucocorticoid is dexamethasone.

7. Combination according to claim 6, in which said superantagonist is the protein called SANT-7 and said glucocorticoid is dexamethasone.

8. A medicament comprising the combination of claim 1.

9. A method of treatment of tumours comprising administering an effective amount of a medicament of claim 8 to a human in need thereof.

10. The method according to claim 9, in which said tumours are interleukin-6-dependent tumours.

11. The method according to claim 10, in which said tumours are haematological tumours.

12. The method according to claim 11, in which said tumours are multiple myclomas.

13. The method according to claim 12, in which said combination consists of SANT-7 according to claim 4 and dexamethasone.

14. The method according to claim 9, in which said medicament is used in combination with other known medicaments used for the treatment of said tumours.

15. The method according to claim 14, in which said known medicament is a medicament whose active ingredient is of the biological type.

16. The method according to claim 15, in which said active ingredient is an anti-interleukin-6 antibody.

17. The method according to claim 14, in which said known medicament is a medicament whose active ingredient is a chemotherapeutic agent.

18. The method according to claim 17, in which said chemotherapeutic agent is selected from the group consisting of alkylating agents, all-transretinoic acid, thalidomide and biphosphonates.

19. The method according claim 18, in which said biphosphonate is zoledronic acid.

20. The method according to claim 17, in which said chemotherapeutic agent is used at a high dosage in combination with autologous transplantation with stem cells.

21. Pharmaceutical composition containing the combination according to claim 1 in a mixture with at least one pharmaceutically acceptable vehicle and/or excipient.

22-30. (canceled)


SEQUENCE LISTING (4 sequences)

SEQ ID NO: 1 - protein, 184 aa, Homo sapiens
PVPPGEDSKDVAAPHRQPLTSSERIDKQIRDILDFISALRKETCNKSNMCESSKEALAENNLNLPKMAEKDGCFQSGFNEETCLVKIITGLLEFEVYLEYLQNRFESSEEQARAVQMRTKDLIQFLQKKAKNLDAITTPDPTTNASLLTKLQAQNQWLQRMTTHLILRSFKEFLQSSLRALRQM

SEQ ID NO: 2 - protein, 184 aa, Homo sapiens
PVPPGEDSKDVAAPHRQPLTSSERIDKQIRDILDFISALRKETCNKSNMCESSKEALAENNLNLPKMAEKDGCFQSGFNEETCLVKIITGLLEFEVYLEYLQNRFESSEEQARAVQMRTKDLIQFLQKKAKNLDAITTPDPTTNASLLTKLQAQNQRLQRMTTHLILRSFKEFLQSSLRALRQM

SEQ ID NO: 3 - protein, 184 aa, Homo sapiens
PVPPGEDSKDVAAPHRQPLTSSERIDKQIRDILDFISALRKETCNKSNMCESSKEALAENNLNLPKMAEKDGCFQSGFNEETCLVKIITGLLEFEVYLEYLQNRFESSEEQARAVQMRTKDLIQFLQKKAKNLDAITTPDPTTNASLLTKLQAQNQWLQDMDTHLILRSFKEFLQSSLRALRQM

SEQ ID NO: 4 - protein, 184 aa, Homo sapiens
PVPPGEDSKDVAAPHRQPLTSSERIDKQIRDILDFISALRKETCNKSNMCESSKEADAFWNLNLPKMAEKDGCFYKGFNEETCLVKIITGLLEFEVYLEYLQNRFESSEEQARAVQMRTKDLIQFLQKKAKNLDAITTPDPTTNASLLTKLQAQNQWLQDMTTHLILRSFKEFLIRSLRALRAM